(19) United States
(12) Patent Application Publication
(10) Pub. No.: US 2022/0040697 A1
(43) Pub. Date: Feb. 10, 2022

(54) CELL ANALYSIS SYSTEMS

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 17/416,430

(22) PCT Filed: Feb. 1, 2019
(86) PCT No.: PCT/US2019/016342

§ 371 Date: Jun. 18, 2021

(51) Int. Cl. B01L 3/00; G01N 33/50; G01N 27/04

(52) U.S. Cl. CPC B01L 3/502761; G01N 33/5005; B01L 2200/0647; B01L 2300/0627; G01N 27/04

(57) ABSTRACT

In one example in accordance with the present disclosure, a cell analysis system is described. The cell analysis system includes a substrate. Formed in the substrate is a feedback-controlled lysis system to rupture a cell membrane. The feed-back-controlled lysis system includes at least one lysing chamber to receive a single cell to be lysed. A lysing element of the feedback-controlled lysis system agitates the single cell and a sensor detects a state within the lysing chamber. The cell analysis system also includes a microfluidic channel formed in the substrate to 1) serially feed individual cells from a volume of cells to the feedback-controlled lysis system and 2) deliver a lysate of a ruptured cell to at least one analysis chamber. The cell analysis system also includes at least one analysis chamber formed in the substrate to process the lysate and a controller to determine when a cell membrane has ruptured.

### BACKGROUND

In analytic chemistry, scientists use instruments to separate, identify, and quantify matter. Cell lysis is a process of rupturing the cell membrane to extract intracellular components for purposes such as purifying the components, retrieving deoxyribonucleic acid (DNA), ribonucleic acid (RNA), proteins, polypeptides, metabolites, or other small molecules contained therein, and analyzing the components for genetic and/or disease characteristics. Cell lysis bursts a cell membrane and frees the inner components. The fluid resulting from the bursting of the cell is referred to as lysate.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a cell analysis system, according to an example of the principles described herein.

FIG. 2 is a diagram of a cell analysis system, according to an example of the principles described herein.

FIG. 3 is a flowchart of a method for cell analysis, according to an example of the principles described herein.

FIG. 4 is a block diagram of a cell analysis system, according to another example of the principles described herein.

FIG. 5 is a diagram of a component controller, according to an example of the principles described herein.

FIG. 6 is a flowchart of a method for cell analysis, according to an example of the principles described herein.

FIG. 7 is a diagram of a cell analysis device, according to an example of the principles described herein.

FIG. 8 is a diagram of a cell analysis device, according to an example of the principles described herein.

FIG. 9 is a diagram of a cell analysis device, according to an example of the principles described herein.

FIG. 10 is a diagram of a cell analysis device, according to an example of the principles described herein.

FIG. 11 is a diagram of a cell analysis device, according to an example of the principles described herein.

FIG. 12 is a diagram of a cell analysis device, according to an example of the principles described herein.

FIG. 13 is a diagram of a cell analysis device, according to an example of the principles described herein.

FIG. 14 is a diagram of a cell analysis device, according to an example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Cellular analytics is a field of chemistry that uses instruments to separate, identify, and quantify matter. Much information can be collected from a cellular sample. For example, the mechanical properties of the cell membrane and even more specifically information relating to the mechanical breakdown of the cell membrane can provide insight to the characteristics and state of a cellular sample. For example, in some cases the physical characteristics of a particular cell can be used to classify and/or differentiate the particular cell from other cells. In another example, changes to the physical characteristics of a cell can be used to determine a state of the cell. For example, parasitic invasion of a cell—such as occurs in cells affected by malaria—can alter the membrane of the cell. Gross changes to tissue, such as when cancer is present in a cell, can also alter the physical properties of the cell membrane. In other words, cell membrane strength indicates cell membrane composition and cell composition. Accordingly, a cell analysis system that can measure cell membrane strength provides to an individual, information regarding the cell membrane behavior, from which characteristics of the cell can be determined.

The intracellular components of the cell also provide valuable information about a cell. Cell lysis is a process of extracting intracellular components from a cell and can also provide valuable information about a cell. During lysis, the intracellular components are extracted for purposes such as purifying the components, retrieving DNA and RNA proteins, polypeptides, metabolites, and small molecules or other components therein, and analyzing the components for genetic and/or disease characteristics. Cell lysis ruptures a cell membrane and frees the inner components. The fluid containing the inner components is referred to as lysate. The contents of the cell can then be analyzed by a downstream system.

The study and analysis of the lysate of a cell provides information used to characterize and analyze a cell. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

While cellular analytics is useful, refinements to the operation may yield more detailed analysis results. For example, in general it may be difficult to obtain a correlation between 1) the mechanical and chemical properties of a cell and 2) the genetic information of the cell. That is, a user cannot simultaneously get mechanical and genetic information from a single sample. To get both genomic and mechanical information, two different samples would be used. However, as the different samples may have different properties, any correlation between the separately collected genomic and mechanical information would rely on a similarity between the two samples, which similarity may not exist or may be tenuous.

Accordingly, a scientist may have to pick from between the two pieces of information (e.g., mechanical and genomic), which they would like to collect. It may be more desirable to obtain the genomic information from the cell as it provides more information. However as described above, the mechanical properties of a cell also provide valuable information. For example, lysis information allows a user to infer cell mechanical properties which may indicate to the user the state of the cell, i.e., dead/living, diseased/healthy.

Moreover, knowing the correlation between mechanical and genetic characteristics of a cell can have many advantages. For example, a correlation between genomic information and a cells susceptibility to lysis may allow a prediction of lytic antibiotic resistance of a cell based on the cell's genetic information. In another example, this correlation is relevant in situations where, for example, cell membrane properties play a role in disease pathology. For example, the elasticity (mechanical property) of a circulating tumor cell may be a determining factor of the cell's metastatic potential and therefore may be an indicator of cancerous cells. In this example, the genetic information collected form a sample indicates what mutations are activated in the cell and may indicate which pathways are up or down regulated. From the genetic and mechanical information, a medical professional may determine which chemotherapy to prescribe as the role of many chemotherapeutics is to affect these pathways. As yet another example, malaria, which is a parasitic infection of red blood cells that changes a stiffness (mechanical property) of the red blood cells and changes the transportation of these cells through the circulatory system. By obtaining the genetic information at the same time, a scientist may determine a type of parasite (there are many malarial parasites for example) that are affecting the patient. With such detailed solutions, a more specific anti-malarial process may be followed. Accordingly, both pieces of information, i.e., mechanical properties and genetic information, for a cell are valuable and useful in analytic chemistry.

Still further cell populations are heterogeneous, meaning each cell in a population may be different from others and may have different responses and characteristics. As a specific example, a sample, such as a blood sample, may include a number of different kinds of cells, each to perform different function and different in its physical and chemical makeup. This heterogeneity of a sample is a building block of the foundations of sustainable life. For example, the different cells in blood allow the blood to sustain human life. Accordingly, when a cell sample is analyzed, it may be desirable to individually analyze the cells in a population, specifically their nucleic acid profiles.

Accordingly, the correlation between mechanical and genetic information may also be heterogeneous. Accordingly, it is not only desirable to obtain both pieces of information, but it may be desirable to obtain genomic and mechanical properties at a single cell level so as to remove inter-sample variation from any resulting correlation.

While some solutions have been presented, they are inadequate for any number of reasons. For example, flow cytometry is an example of a single cell analysis technique. Flow cytometry differentiates cells based on their spatial scattering profile or their bulk fluorescence. However, flow cytometry does not obtain genetic and/or mechanical information about a cell. To obtain the mechanical properties of a cell, deformation flow cytometry may be performed which combines differentiation based on fluorescence and scattering with cell deformation behavior. However, deformation flow cytometry does not obtain genetic information.

In some cases, single cell genomic analysis may be performed. In this example, cell solutions are diluted and aliquoted into wells. The wells are lysed indiscriminately and after certain preparation operations, the genetic material is sequenced. However, this does not indicate any mechanical information about the cell nor of the cell membrane.

The present specification provides for such an individual cell lysis and multi-modal analysis. Specifically, the present specification describes a system for simultaneously obtaining genetic (RNA, DNA) information and cell membrane strength (chemical and/or mechanical) information of a cell population with single cell resolution in an automated fashion on a large number of cells. The system includes a reservoir that holds a cell suspension, a flow structure that segregates the cells such that they enter a lysing chamber one at a time. Within the lysing chamber a feedback-controlled lysing operation is carried out. Information regarding the lysing operation and the properties of the lysate are passed to a controller to analyze the cell based on both pieces of information. The present specification also describes a nucleic acid analysis system, all on the same substrate. Accordingly, valuable information from precious cell populations and each individual cell can be made even when the number of cells to be analyzed cannot be increased.

In addition to these components, the system includes the downstream analysis devices that operate on the lysate. That is, not only is the cell prepared on a single substrate, it is also analyzed on that same substrate. In other words, a cell may pass from a reservoir throughout an entire analysis path, i.e., lysis, lysate analysis, membrane rupture analysis, without ever being touched by human hands and in some cases without being exposed to the environment. The reduced amount of user manipulation decreases the likelihood of user error during cellular analysis and the lack of exposure to the environment reduces the likelihood of contamination of the cell, which obviously results in more accurate test results.

Specifically, the present specification describes a cell analysis system that includes a substrate and a feedback-controlled lysis system formed in the substrate to rupture a cell membrane. The feedback-controlled lysis system includes 1) at least one lysing chamber to receive a single cell to be lysed, 2) a lysing element to agitate the single cell, and 3) a sensor to detect a state within the lysing chamber. The cell analysis system also includes a microfluidic channel formed in the substrate to 1) serially feed individual cells from a volume of cells to a feedback-controlled lysis system and 2) deliver a lysate of a ruptured cell to at least one analysis chamber. At least one analysis chamber of the cell analysis system is also formed in the substrate and is to process the lysate. A controller of the cell analysis system determines when a cell membrane has ruptured.

The present specification also describes a method. According to the method, a cell to be lysed is received at a feedback-controlled lysis system of a cell analysis system. The feedback-controlled lysis system is activated to agitate the cell to be lysed. It is detected when a cell membrane is ruptured and responsive to such detection, a lysate of the cell is transported to an analysis chamber. An analysis operation is then performed on the lysate.

In another example, the cell analysis system includes a substrate and at least one cell analysis device formed in the substrate. Each cell analysis device includes a feedback-controlled lysis system to rupture a cell membrane. The feedback-controlled lysis system includes 1) at least one lysing chamber to receive a single cell to be lysed, 2) a lysing element to agitate the single cell, and 3) a sensor disposed in the lysing chamber to detect a state within the lysing chamber. Each cell analysis device also includes a microfluidic channel to 1) serially feed individual cells from a volume of cells to a feedback-controlled lysis system and 2) deliver a lysate of a ruptured cell to at least one analysis chamber. Each cell analysis device also includes at least one analysis chamber to process the lysate, a cell reservoir to hold a volume of cells, and a pump to move fluid through the cell analysis system. A controller of the cell analysis system is disposed on the substrate and analyzes the cell and includes 1) a lysate analyzer to analyze properties of a lysate of the cell, 2) a rupture analyzer determines the rupture threshold of the cell based on parameters of a cycle when a cell membrane ruptures, and 3) a component controller to alter operation of at least one component of the analytic device based on a cell rupture.

In summary, using such a cell analysis system 1) provides for effective monitoring of cell lysis on a per-cell basis; 2) ensures sufficient lysis without degradation to cell contents; 3) provides control of the amount of analyte to be delivered downstream; 4) identifies subsets of cell population that are difficult to lyse; 5) provides a feedback signal for automated control of the lysis operation, 6) allows combined cell analysis, i.e., a genetic analysis and a mechanical property analysis; 7) allows for an entire preparation and analysis operation on a single substrate; 8) can be integrated onto a lab-on-a-chip; 9) is scalable and can be parallelized for high throughput, 10) is low cost and effective, and 11) reduces the contamination of the sample. However, the devices disclosed herein may address other matters and deficiencies in a number of technical areas.

As used in the present specification and in the appended claims, the term “cell membrane” refers to any enclosing structure of a cell, organelle, or other cellular particle.

Further, as used in the present specification and in the appended claims, the term “agitation cycle” refers to a period when a cell is exposed to the operations of a lysing element. For example, an agitation cycle may refer to each time a cell is looped past a single lysing element. In another example, a cell passes through an agitation cycle each time it passes by a lysing element in a string of multiple lysing elements.

Even further, as used in the present specification and in the appended claims, the term “rupture threshold” refers to the amount of stress that a cell can withstand before rupturing. In other words, the rupture threshold is the threshold at which the cell ruptures. The rupture threshold may be determined based on any number of factors including a number of agitation cycles a cell is exposed to and the intensity of the agitation cycles.

Yet further, as used in the present specification and in the appended claims, the term “parameters” refers to the operating conditions in a particular agitation cycle. For example, a “parameter” may refer to a type of lysing element and/or a lysing strength. For example, agitation parameters for an agitation cycle may include whether a lysing element is a thermal inkjet resistor, a piezo-electric device, or an ultrasonic transducer. Agitation parameters also refer to the operating conditions of the particular lysing element. For example, the parameters of an ultrasonic transducer may refer to the frequency, amplitude (power), and/or phase of ultrasonic waves. The parameters of the thermal inkjet resistor and piezo-electric device may refer to the size of the element and/or the voltage, a pulse energy, and a pulse duration applied to the element.

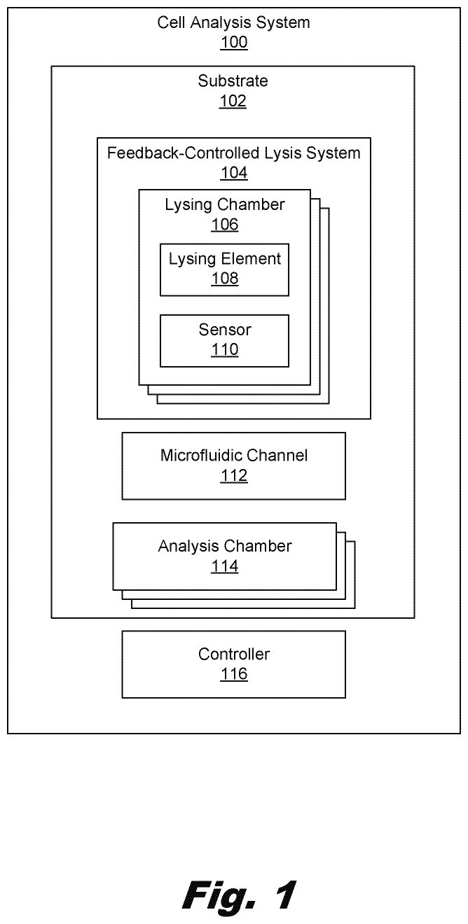

Turning now to the figures, FIG. 1 is a block diagram of a cell analysis system (100), according to an example of the principles described herein. In some examples, the cell analysis system (100) is part of a multi-function chip, which may be referred to as a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small-scale fluid analysis functionality.

In other words, the components, i.e., the feedback-controlled lysis system (104), lysing chamber(s) (106), microfluidic channel (112), and analysis chamber(s) (114) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The cell analysis system (100) includes a substrate (102) on which the other components of the cell analysis system (100) are disposed. The substrate (102) may be formed of any rigid material, such as plastic or silicon, that has the other components, i.e., the lysing chamber (106), lysing element (108), sensor (110), microfluidic channel (112), and analysis chambers (114) disposed on top or within.

Disposed on, or in, the substrate (102) is a feedback-controlled lysis system (104) which performs the cellular analysis. In general, lysis refers to the agitation of a cell with the objective of rupturing a cell membrane. Lysis ruptures a cellular particle membrane and frees the inner components. The fluid containing the inner components is referred to as lysate. The contents of the cellular particle can then be analyzed by a downstream system.

To carry out such lysis, each feedback-controlled lysis system (104) includes a variety of sub-components. Specifically, each feedback-controlled lysis system (104) includes at least one lysing chamber (106) to receive a single cell to be lysed. That is, the lysing chamber (106) may receive the cells single-file, or serially. Thus, lysing operations can be performed on a single cell and that cell's particular properties may be analyzed and processed.

In some examples, the lysing chamber(s) (106) may be no more than 100 times a volume of a cell to be lysed. In other examples, the lysing chamber (106) may have a cross-sectional size comparable with the cell size and in some cases smaller than the cell so as to deform the cell before or during the rupturing of the cell membrane. That is, the lysing chamber (106) may be a microfluidic structure.

Each lysing chamber (106) includes a lysing element (108) to agitate the single cell. The lysing element (108) may implement any number of agitation mechanisms, including shearing, localized heating, and shearing by constriction. In another example, repeated cycles of freezing and thawing can disrupt cells through ice crystal formation. Solution-based lysis is yet another example. In these examples, the osmotic pressure in the cellular particle could be increased or decreased to collapse the cell membrane or to cause the membrane to burst. As yet another example, the cells may be forced through a narrow space, thereby shearing the cell membranes.

In one example, the lysing element (108) is a thermal inkjet heating resistor disposed within the lysing chamber (106). In this example, the thermal inkjet resistor heats up in response to an applied current. As the resistor heats up, a portion of the fluid in the chamber vaporizes to generate a bubble. This bubble generates a pressure and shear spike which ruptures the cell membrane.

In another example, the lysing element (108) may be a piezoelectric device. As a voltage is applied, the piezoelectric device changes shape which generates a pressure pulse in the chamber that generates a pressure and shear spike which ruptures the cell membrane.

In yet another example, the lysing element (108) may be a non-reversible electroporation electrode that forms nano-scale pores on the cell membrane. These pores grow and envelope the entire cell membrane leading to membrane lysis. In yet another example, the lysing element (108) is an ultrasonic transducer that generates high energy sonic waves. These high energy waves may travel through the wall of the chamber to shear the cells disposed therein.

The different types of lysing elements (108) each may exhibit a different agitation mechanism. For example, the agitation mechanism of an ultrasonic transducer is the ultrasonic waves that are emitted and that shear the cells. The agitation mechanism of the thermal inkjet heating resistor is the vapor bubble that is generated and ruptures the cell membrane. The agitation mechanism of the piezo-electric device is the pressure wave that is generated during deformation of the piezo-electric device, which pressure wave shears the cell membrane. While particular examples of lysing elements (108) have been described herein, a variety of lysing element (108) types may be implemented in accordance with the principles described herein.

A feedback-controlled lysis system (104) refers to a system wherein the lysing element (108) operation is monitored to ensure lysis occurs as desired. That is, the feedback provides a quality control check over a lysing operation. In this example, the lysing chamber (106) includes a sensor (110) to determine a state within the lysing chamber (106), and more specifically to determine when a cell has ruptured, and to return the cell to within range of the lysing element (108) in the case the cell has not ruptured. That is, the sensor (110) detects a change in the cell based on an agitation of the cell by the lysing element (108). If no change is detected, the cell is kept in, or returned to, the lysing chamber (106) for another agitation cycle. Accordingly, rather than activating the lysing element (108) and hoping that lysing occurs, a feedback-controlled lysis includes a sensor (110) to ensure lysing occurs prior to further processing of the lysate.

The sensor (110) may take many forms. For example, the sensor (110) may be an optical scatter sensor that determines cell rupture based on a scatter of reflected energy waves. The sensor (110) may be an optical fluorescence sensor that detects cell rupture based on the detection of certain fluorescent markers. In other examples, the sensor (110) may be an optical bright field sensing system, an optical dark field sensing system, or a thermal property sensor.

In one particular example, the sensor (110) is an impedance sensor. Specifically, the sensor (110) may include at least one pair of electrodes spaced apart from one another by a gap. These electrodes detect a level of conductivity within the gap. That is, incoming cells to a lysing chamber (106), and the solution in which they are contained, have a predetermined electrical conductivity. Any change to the contents within the lysing chamber (106) will effectively change the electrical conductivity within the lysing chamber (106). Specifically, as the cells are ruptured and the nucleic acid pours out, the conductivity would increase. To measure the conductivity, a resistance of solution between electrodes of the impedance sensor is measured and a conductivity determined therefrom. In some examples, a single pair of electrodes are used, with one electrode plate placed at either end of a chamber. In another example, multiple pair of electrodes are used. For example, one pair of electrode plates could be placed at the inlet and another pair of electrode plates placed at the outlet.

The cell analysis system (100) includes a microfluidic channel (112) that transports the cells and lysate throughout the cell analysis system (100). The microfluidic channel (112) is coupled at one end to a cell reservoir and serially feeds individual cells from the volume of cells to the feedback-controlled lysis system (104).

The microfluidic channel (112) may have properties such that cells are passed individually thus facilitating per-cell lysis and analysis. Such a serial, single-file introduction of cells into the lysing chamber (106) may be facilitated by a microfluidic channel (112) having a cross-sectional area size on the order of the cell diameter. Following lysis, the microfluidic channel (102) delivers a lysate of the ruptured cell to at least one analysis chamber (114).

The at least one analysis chamber (114) is also formed in the substrate (102) and processes the lysate. That is, the at least one analysis chamber (114), like the lysing chamber(s) (106) and the microfluidic channel (112), may be embedded in the substrate (102) such that the entire preparation (lysis) and analysis may occur on the same substrate without having to be handled by users and thus also preventing environmental contamination. In general, a variety of analysis tests may be carried out in the analysis chamber (114). For example, the lysate may be combined and mixed with a master mix, which may include primers. In some examples, the analysis chamber(s) (114) may include a heater to change the temperature of the lysate, which may be desirable in certain analytic operations.

Any number of analysis operations may be carried out in the analysis chamber (114). For example, a polymerase chain reaction (PCR) operation may be carried out to amplify a segment of nucleic acid such that additional tests may be carried out on the nucleic acid. In another example, the analysis may be a genetic test itself. Other examples of analysis operations that could be performed in the analysis chambers (114) include nucleic acid hybridization, and antigen antibody hybridization. In other words, the number of genetic analysis operations carried out in the analysis chamber (114) are of a wide variety. Accordingly, any solution that is combined with the lysate in the analysis chamber (114) may be of a variety of types. For example, for gene amplification, the solution may include the components for DNA amplification. Specifically, the solution may include polymerase and a buffer.

As described above, by embedding the analysis chamber (114) on the substrate (102) a variety of cell analytic operations, lysing and analysis, are performed at a single location without having to transport the cell. In some cases, cell structure may change quickly overtime. Accordingly, by including the lysis system (104) and the analysis chambers (114) on the same structure, less time passes between these operations, thus reducing scenarios where cell changes between lysing and analysis skew results. This result may therefore be particularly useful for analysis of short-lived RNA molecules.

The cell analysis system (100) also includes a controller (116) to analyze a ruptured cell. Specifically, the controller (116) determines when the cell membrane has ruptured based on an output of the sensor (110). As a specific example of an impedance sensor, the controller (108) may compare detected levels of conductivity within the lysing chamber (106) with a threshold level of conductivity associated with a ruptured cell. Accordingly, once the detected level of conductivity within the lysing chamber (106) has reached the threshold value, the controller (116) may determine that a cell has been ruptured.

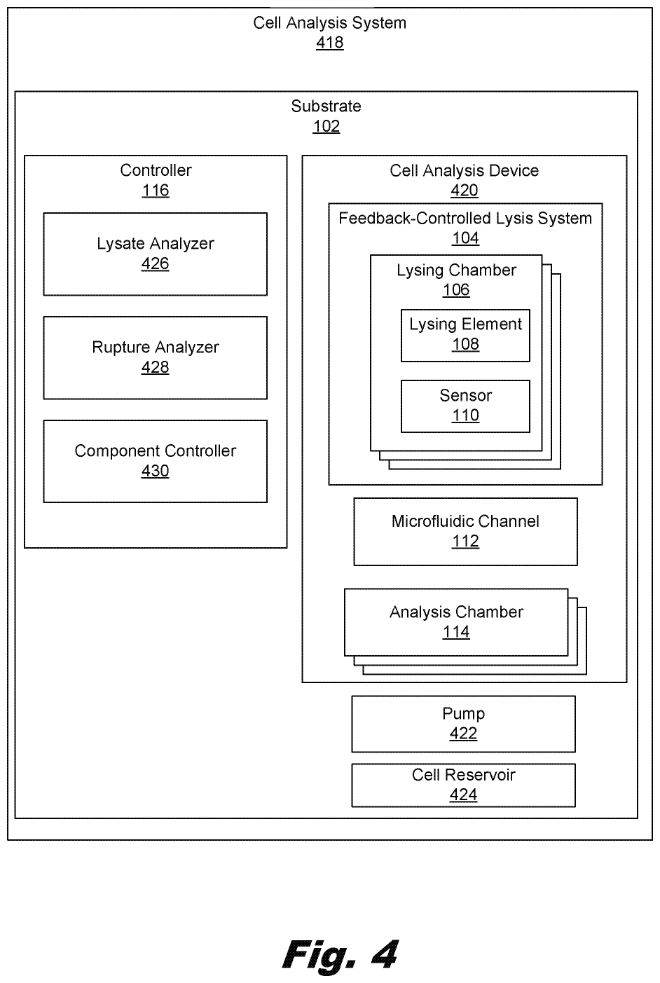

The controller (116) also determines a rupture threshold for the cell. That is, a notification of each agitation cycle may be passed to the controller (116). Accordingly, by knowing the number of agitation cycles, the strength of each agitation cycle, a type of lysing element (108) used for each agitation cycle, and the point at which the cell ruptures (as determined by the sensor (110)), the controller (116) can determine the rupture threshold of the cell. This information, i.e., the mechanical strength of a cell, may be used by the controller (116) or passed to a downstream system for further analysis of the particular cell. As depicted in FIG. 1, in some examples, the controller (116) is not disposed on the substrate (102). However, it may be the case as depicted in FIG. 4, that the controller (116) is disposed on the substrate (102).

As described herein, the present specification describes a cell analysis system (100) that, on a single embedded substrate (102), monitors the lysis operation and analyses the resulting lysate. Such a cell analysis system (100) provides a simple, scalable solution for effectively lysing and analyzing cell pathways. Such information is useful in a wide variety of applications.

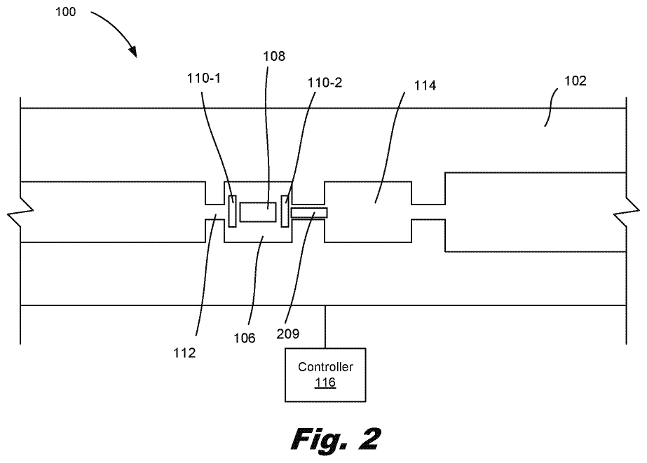

FIG. 2 is a diagram of a cell analysis system (100), according to an example of the principles described herein. As described above, the cell analysis system (100) includes a microfluidic channel (112) that gates the serial introduction of cells to be lysed into a lysing chamber (106). There, action of a lysing element (108), after any number of agitation cycles, ruptures the cell membrane, pouring out a lysate. The moment of such lysis is detected by the sensors (110-1, 110-2) which as depicted in FIG. 2, may be disposed within the lysing chamber (106). If lysis has not occurred, the cell may be returned to the lysis chamber (106) via the action of a return pump (209). The return pump (209) may be activated by the controller (116) based on a signal received form the sensor (110) that a cell has not ruptured. The lysate is then passed via the microfluidic channel (102) to an analysis chamber (114) where any number of nucleic acid tests can be performed. A non-exhaustive list includes PCR, DNA and RNA sequencing, lysate protein detection, antibody detection, and hybridization tests. While a few specific references are made to particular tests, any number of nucleic acid tests could be facilitated in the analysis chamber (114).

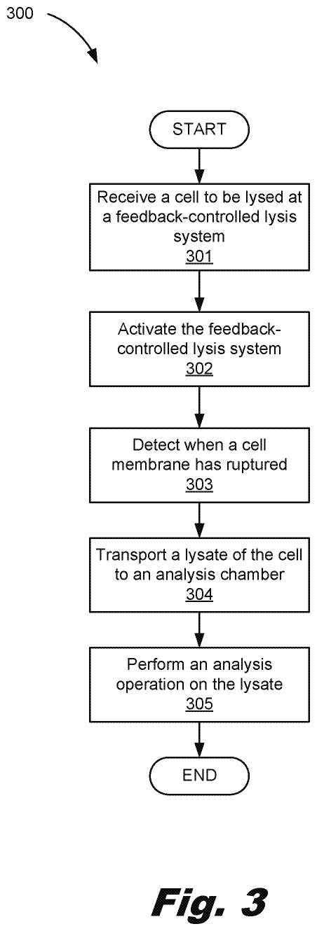

FIG. 3 is a flowchart of a method (300) for cell analysis, according to an example of the principles described herein. According to the method (300), a cell to be lysed is received (block 301) at a feedback-controlled lysis system (FIG. 1, 104). That is, a fluid that contains a cell is transported into the lysing chamber (FIG. 1, 106) via a microfluidic channel (FIG. 1, 112). In some examples this is done by activating a pump which is integrated into a wall of the microfluidic channel (FIG. 1, 112).

According to the present method (300), the volume of cells are serially received (block 301) at the feedback-controlled lysis system (FIG. 1, 104). That is, each cell within the sample may be received (block 301) one at a time. This is done by the microfluidic channel (FIG. 1, 112) which that gates introduction of one cell at a time into the lysing chamber (FIG. 1, 106). Such single-file, or serial, inlet of cells facilitates an individual analysis of cells. Accordingly, rather than analyzing a portion of the sample and extrapolating therefrom, each cell of the sample may be analyzed. Thus, a complete analysis of the sample is performed. As described above, the cell analysis system (FIG. 1, 100) may include any number of feedback-controlled lysis systems (FIG. 1, 104) and analysis chambers (FIG. 1, 114). Thus, while each cell analysis system (FIG. 1, 100) operates on a single cell, a die with multiple cell analysis systems (FIG. 1, 100) may analyze the cells in parallel. Doing so may increase throughout.

With a cell present in a corresponding lysing chamber (FIG. 1, 106), a feedback-controlled lysis system (FIG. 1, 104), and more specifically the lysing element (FIG. 1, 108), is activated (block 302) to agitate the cell to be lysed. As described above, lysing is an operation wherein a cell is agitated until its membrane ruptures or otherwise breaks down. The point at which a cell membrane breaks down may be referred to as a rupture threshold and may provide valuable information about a particular cell as described above.

As described above, the lysing in the present method (300) is feedback-controlled. That is, in some examples, a lysing element (FIG. 1, 108) may not rupture a cell membrane. For example, the cell membrane may be robust against a particular intensity of agitation. Without feedback-controlled lysis, the cell may leave the lysing chamber intact. Outputting an intact cell when a lysed cell is desired and/or expected, may result in skewed results.

Accordingly, it is detected (block 303) when a cell membrane is ruptured. Such information may be used to 1) allow the lysate to proceed down the fluidic path and 2) characterize the cell. Accordingly, when a sensor (FIG. 1, 110) indicates that, despite the operations of the lysing element (FIG. 1, 108), the cell membrane has not ruptured, the controller (FIG. 1, 116) may activate a return pump (FIG. 2, 209) to return the cell to be under the influence of the lysing element (FIG. 1, 108). In this example, a second lysing cycle, either at the same intensity or an increased intensity, may be executed such that the cell may be ruptured. That is, in some examples agitation intensity may be incrementally adjusted until the cell membrane ruptures.

In another example, rather than activating a return pump (FIG. 2, 209), the controller (FIG. 1, 116) may activate a downstream lysing element (FIG. 1, 108). In this example, the second lysing element (FIG. 1, 108) may be at the same or increased intensity.

In either case, once in the feedback-controlled lysis system (FIG. 1, 104), the cell is exposed to repeated agitation cycles. The lysing intensity may increase or remain the same. For example, a single lysing element (FIG. 1, 108) may be adjustable. As specific examples, the energy applied to a thermal inkjet resistor may increase or the intensity of ultrasonic waves may increase with each agitation cycle. In another example, the lysing element (FIG. 1, 108) may have a fixed intensity. In yet another example, the feedback-controlled lysis system (FIG. 1, 104) includes multiple lysing elements (FIG. 1, 108) each with fixed, but differing strengths along a flow path. In any case, the cell is exposed to multiple agitation cycles until the cell ruptures. As described above, cell rupture may be determined by the sensor (FIG. 1, 110). That is, the sensor (FIG. 1, 110), whatever type it may be, can detect the difference between an intact cell and a ruptured cell. Accordingly, such a feedback-controlled lysing operation ensures that a cell that is intended to be lysed is in fact lysed.

Once lysed, the lysate is transported (block 304) to an analysis chamber (FIG. 1, 114) on the same microfluidic substrate (FIG. 1, 102) where an analysis operation is performed (block 305) on the lysate. As described, any type of analysis may be performed on the cell in the analysis chamber (FIG. 1, 114) including, but not limited to polymerase chain reactions (PCRs), DNA and RNA sequencing, protein detection and analysis, antibody analysis, and a variety of hybridization reactions. Accordingly, the method described herein provides for sample preparation and analysis in a system that is embedded on a single substrate (FIG. 1, 102) so as to prevent environmental contamination and reduce user interference which may lead to measurement error.

FIG. 4 is a block diagram of a cell analysis system (418), according to an example of the principles described herein. In some examples, the cell analysis system (418) is part of a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small-scale fluid analysis functionality.

In other words, the components, i.e., the cell analysis device(s) (420), feedback-controlled lysis system (104), microfluidic channel (112), analysis chamber (114), pump (422), and cell reservoir (424) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The cell analysis system (418) include at least one cell analysis device (420). The cell analysis device (420) refers to the components that perform multiple operations on a cell. As described above, the cell analysis device (420) and the components that make up the cell analysis device (420) are formed on a single substrate (102). In some examples as depicted in FIG. 4, the controller (116) is also integrated on the same substrate (102). Thus, this one substrate (102) holds each component for cell analysis as well as the circuitry that controls the cell analysis operations. That is, the present cell analysis system (418) facilitates the complete analysis of a cell, at a single cell resolution, on a single physical structure. The substrate (102) may be formed of any material including plastic and silicon, such as in a printed circuit board.

The cell analysis device (420) includes the microfluidic channel (112) that 1) serially feeds individual cells to a feedback-controlled lysis system (104) and that delivers a lysate of a ruptured cell to at least one analysis chamber (114).

The cell analysis device (420) also includes the feedback-controlled lysis system (104) to rupture a cell membrane. The feedback-controlled lysis system (104) includes at least one lysing chamber (106) to receive a single cell to be lysed, a lysing element (108) to agitate the single cell, and a sensor (110) to detect a state within the lysing chamber (106). The cell analysis device (420) also includes the analysis chambers (114) where lysate processing occurs. As noted above, the analysis chambers (114) are disposed on the same substrate (102) as the feedback-controlled lysis systems (104) such that lysis and analysis occur on one physical device that is connected via the microfluidic channel (112).

The cell analysis device (420) may include additional components such as a cell reservoir (424) to hold a volume of cells and a pump (422) to move fluid through the cell analysis device (420). In some examples, the pump (422) may be an integrated pump, meaning the pump (422) is integrated into a wall or substrate floor of the microfluidic channel (112). In some examples, the pump (422) may be an inertial pump which refers to a pump (422) with an asymmetric position within the microfluidic channel (112). The asymmetric positioning within the microfluidic channel (112) facilitates an asymmetric response of the fluid to the pump (422). The asymmetric response results in fluid displacement when the pump (422) is actuated. In some examples, the pump (422) may be a thermal inkjet resistor, or a piezo-drive membrane or any other displacement device.

The cell analysis system (418) also includes a controller (116) that analyzes the cells of the sample. As depicted in FIGS. 1 and 4, the controller (116) may be integrated on the substrate (102) or may be separate, and in some cases external to, the substrate (102). The controller (116) includes various components to make such an analysis. First, the controller (116) includes a lysate analyzer (426) to analyze properties of a lysate of the cell. That is, after the cell has been ruptured, the contents therein may be analyzed and information provided to the lysate analyzer (426). A variety of pieces of information can be collected from the lysate. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

The controller also includes a rupture analyzer (428) which determines a rupture threshold of the cell based on the parameters of the agitation when the cell membrane ruptures. That is, as described above a cell may be exposed to one or multiple agitation cycles. The parameters of the different agitation cycles can be passed to the rupture analyzer (428) which analyzes parameters of an agitation when a cell membrane ruptures. The rupture analyzer (428) may use this information to perform a variety of analytical operations. That is, the rupture analyzer (428) can determine the rupture threshold by knowing how many agitation cycles the cell was exposed to and the intensity of each agitation cycle. Thus, the rupture analyzer (428) determines at what agitation intensity the cell ultimately ruptures. With such information on hand, the rupture analyzer (428) can determine certain properties of the cell including cell type, cell state, etc.

The rupture analyzer (428) may use this information to perform a variety of analytical operations. For example, the rupture analyzer (428) may differentiate cells in a sample based on different rupture thresholds. In this example, the rupture analyzer (428) may receive, for multiple cells, information regarding the results of lysing. Based on the results, the rupture analyzer (428) may determine when each cell in a sample is ruptured. Different types of cells may rupture under different intensities. Accordingly, based on when a cell ruptures, the rupture analyzer (428) may be able to determine the cell type.

As another example, the rupture analyzer (428) may be able to determine a state of a cellular sample. For example, it may be determined that healthy cells rupture at a particular lysing intensity. This may be determined by passing healthy cells through the cell analysis system (418) and collecting rupturing information. Accordingly, a sample to be analyzed may subsequently be passed through the cell analysis system (418) and rupturing information collected for these cells in the sample. If the rupturing information indicates that the sample cells rupture at a lower intensity than the healthy cells, the rupture analyzer (428) may determine that the sample cells are diseased.

As yet another example, the rupture analyzer (428) may be able to differentiate between live cells and dead cells based on the rupturing thresholds of different cells as determined by the cell analysis device (420). That is, live cells may be more robust against lysing and therefore have a higher rupturing threshold as compared to dead cells which may rupture at a lower intensity.

Thus, the present cell analysis system (418) provides a way to collect information related to both the lysate and the mechanical properties of the cell membrane from a single sample. Being able to collect both pieces from a single sample removes any bias resulting from intra-sample variation. For example, both the elasticity of a circulating tumor cell as well as the genetic components of the tumor cell may be determined from a single sample. As yet another example, both a stiffness of a red blood cell as well as the genetic aspects of the cell can be analyzed to determine if the cell is affected by malaria. Being able to collect both pieces of information from a single sample also makes more effective use of the sample. That is, rather than requiring two groups of the sample, one for mechanical testing and one for genetic testing, both pieces of information from one group of the sample.

The controller (116) also includes a component controller (430) to alter operation of at least one component of the cell analysis device (420) based on a cell rupture. For example, the component controller (430) may trigger certain pumps (422), when it is determined that a cell has ruptured.

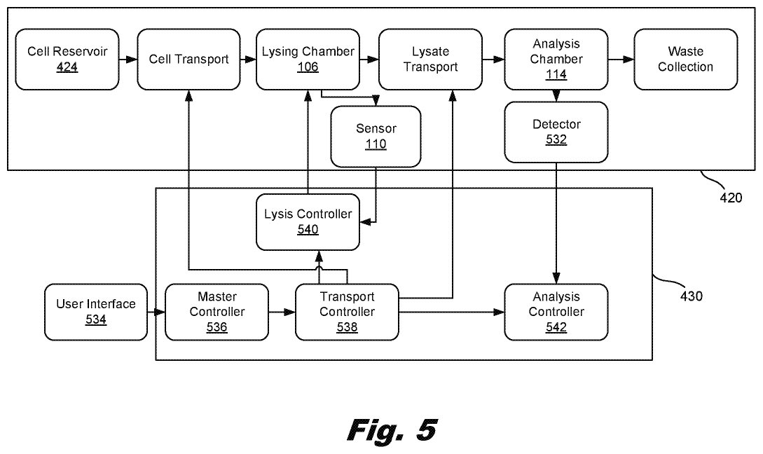

FIG. 5 is a diagram of a component controller (430), according to an example of the principles described herein. As described above, the controller (FIG. 1, 116) includes a component controller (430) that can activate/deactivate different components of the cell analysis device (420) at different times.

In some examples, control over those components is received from a user via a user interface (534). That is, via the user interface (534) a user may set certain lysing and analysis parameters. The master controller (536) of the component controller (430) may provide general control over the entire operation and may communicate information between the user interface (534) and the individual component control devices.

The transport controller (538) controls the transport of the cell and lysate throughout the cell analysis device (420). For example, as described above, the cell analysis device (420) may include a pump (FIG. 4, 422) that moves the solution along the path from the cell reservoir (424) to the lysing chamber (106) and in some cases may include a return pump (FIG. 2, 209) to return an un-lysed cell to the lysing chamber (FIG. 1, 106). The transport controller (538) regulates this transport. That is, the transport controller (538) may activate/deactivate the pump (FIG. 4, 422) at different times to effectuate movement of cells towards the lysing chamber (106).

The transport controller (538) also regulates transport of the lysate, similarly, by activating/deactivating the pump (FIG. 4, 422) or another pump. In this example, control over the lysate transport may be based on the lysis operation. That is, if the sensor (110) detects that a cell rupture has not occurred, lysate transport is halted such that the cell remains in the lysing chamber (106). By comparison, if the sensor (110) detects that a cell rupture has occurred, lysate transport is initiated such that the lysate passes towards the analysis chamber (114).

The lysis controller (540) controls the operation of the lysing elements (FIG. 1, 108), again potentially based on the output of the sensor (110). That is, if the sensor (110) detects that a cell rupture has not occurred, a lysing element (FIG. 1, 108) may be activated, either at the same or additional strength. By comparison, if the sensor (110) detects that a cell rupture has occurred, lysing elements (FIG. 1, 108) may be de-activated so as to prevent over-lysing of the cell and to prevent potential damage to the cell contents.

The analysis controller (542) monitors the operation of the analysis operation. That is, a detector (532) may be set up external to, or disposed within, the analysis chamber (114). This detector (532) can detect conditions within the analysis chamber (114) to determine a state of the analysis, i.e., whether the analysis is complete. The detector (532) can also provide information on the results of the analysis, i.e., what components are present in the lysate, etc. This information is passed to the analysis controller (542) which may regulate devices associated with the cell analysis, and may pass the information through the user interface (534) such that a user can view the results of the analysis. Waste fluid following the analysis can then be collected and properly disposed of.

As a specific example, the detector (532) may detect PCR amplification signals correlating with the presence of a target DNA/RNA sequence. In this example, the detector (532) may be an optical fluorescence detector (532) that detects a fluorescent signal from amplified DNA. Other examples of detectors (532) include electrochemical detectors (532) that detect a redox label, a pH detector (532), and any other type of detector (532) that enables detection of the DNA target presence and amplification. As described above, the detector (532) can be external to the system (for example optical microscope) or integrated in the substrate (102), such as an optical or electrochemical detector (532). This same detector (532) can also be used for antibody detection and another types of analysis.

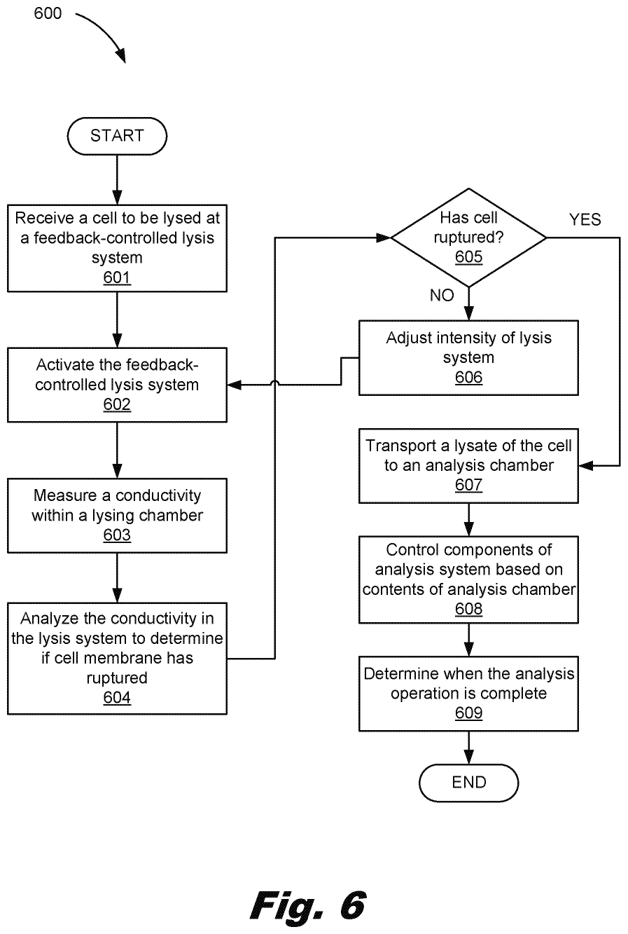

FIG. 6 is a flowchart of a method (600) for cell analysis, according to an example of the principles described herein. According to the method (600), a cell to be lysed is received (block 601) at a feedback-controlled lysis system (FIG. 1, 104) and the feedback-controlled lysis system (FIG. 1, 104) is activated (block 602). These operations may be performed as described above in connection with FIG. 3.

It is then determined whether a cell membrane has ruptured. As described above, this is performed by relying on the sensor (FIG. 1, 110) which in some cases may be an impedance sensor. For example, a conductivity within a lysing chamber (FIG. 1, 106) is measured (block 603) and the conductivity analyzed (block 604) to determine that the cell membrane has ruptured. That is, the measured conductivity value can be used to determine whether a cell wall/membrane has ruptured or not. For example, the controller (FIG. 1, 116) can consult a database with a mapping to determine if the measured level of conductivity is lesser or greater than a value indicative of a threshold lysing. If greater than the threshold value, it may be determined that the cell membrane has ruptured. If not greater than the threshold value, the controller (FIG. 1, 116) may determine that the cell membrane has not ruptured, i.e., if the cell is un-lysed, the cell may be re-lysed.

Specifically, if it is determined that the cell membrane has not ruptured (block 605, determination NO), then a second attempt at lysing may occur. In this second attempt, and for subsequent iterations, the intensity of the lysis may be incrementally adjusted (block 606) until the cell membrane ruptures. Thus, a cell is exposed to increasingly greater strengths of lysis until the cell membrane ruptures. Thus, a precise moment when the cell membrane ruptures is determined.

Such an incremental adjustment may come in a variety of forms. For example, in the case of a single lysing chamber (FIG. 1, 106) and a single lysing element (FIG. 1, 108), the strength of the agitation may increase. For example, if the lysing element (FIG. 1, 108) is an ultrasonic transducer, the frequency and/or amplitude of the waves may be increased. In the example of multiple lysing chambers (FIG. 1, 106) and multiple lysing elements (FIG. 1, 108), the cell may be passed to a downstream lysing chamber (FIG. 1, 106) that includes a lysing element (FIG. 1, 108) with a greater lysing intensity than a previous lysing element (FIG. 1, 108). That is, the lysing elements (FIG. 1, 108) along a flow path may have increasing intensity and therefore a cell that is transported along the flow path is exposed to increased lysing strengths. In either case, single lysing element (FIG. 1, 108) or multiple lysing elements (FIG. 1, 108), the feedback-controlled lysis system (FIG. 1, 104) is re-activated (block 602) and a conductivity measured (block 603) and analyzed (604) to determine a point when the cell ruptures.

If the cell membrane has ruptured (block 605, determination YES), the lysate is transported (block 607) to an analysis chamber (FIG. 1, 114). This may be performed as described in connection with FIG. 3.

According to the method (600), components of the cell analysis device (FIG. 4, 420) are controlled (block 608) based on the contents of the analysis chamber (FIG. 1, 114). For example, as described above, in the analysis chamber (FIG. 1, 114), the lysate may be mixed with a solution to effectuate genetic amplification. However, it may be desirable that this solution is not introduced into the analysis chamber (FIG. 1, 114) until the lysate is also present. Accordingly, the component that is controlled (block 608) based on the presence of the lysate in the analysis chamber (FIG. 1, 114) may be a pump or ejector that delivers the solution.

The method (600) may also include determining (block 609) when the analysis operation is complete. That is, the cell analysis system (FIG. 4, 418) may include a detector (FIG. 5, 532) that monitors the analysis operation and provides feedback to an operator of the experiment. Accordingly, additional control over the processing of a cell is provided via the detection and monitoring of the analysis operation that is occurring in the analysis chamber (FIG. 1, 114).

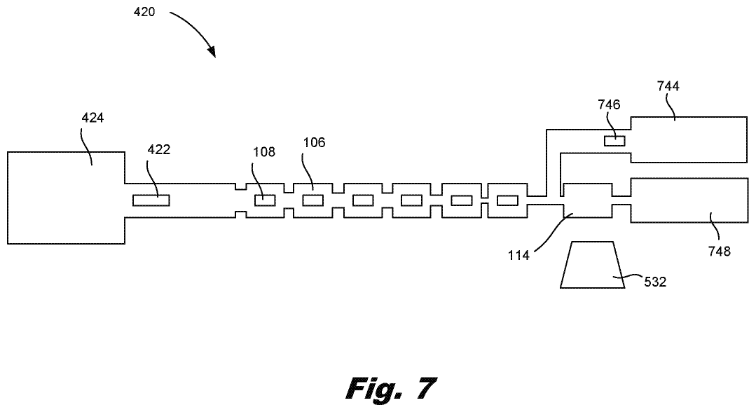

FIG. 7 is a diagram of a cell analysis device (420), according to an example of the principles described herein. FIG. 7 depicts the cell reservoir (424), pump (422), lysing chamber(s) (106) and lysing elements (108) as described above. For simplicity the sensors (FIG. 1, 110) of the system are not depicted in FIG. 7, but as described above are implemented in this and other examples. Specifically, as depicted in FIG. 2, the sensors (FIG. 1, 110) may be placed in each lysing chamber (106) to determine a state within a respective lysing chamber (106). Also, for simplicity, just one instance of a lysing chamber (106) and lysing element (108) are depicted with reference numbers.

In the example depicted in FIGS. 7-14, the feedback-controlled lysis system (FIG. 1, 104) includes multiple sequential lysing chambers (106) and respective lysing elements (108) and sensors (FIG. 1, 110). That is, in the example depicted in FIG. 7, rather than passing a cell by a single lysing element (108) multiple times, the cell is passed by multiple lysing elements (108) a single time. This example may be beneficial in that it alleviates certain controlled elements such as the return pump.

Each sensor (FIG. 1, 110) detects whether a cell rupture occurred in a corresponding lysing chamber (106). In this example, information from each of the sensors (FIG. 1, 110) is passed to the controller (FIG. 1, 116) for cell rupture threshold determination. That is, as a cell passes by each of the sensors (FIG. 1, 110), information is passed to the controller (FIG. 1, 116) to determine whether a corresponding lysing element (108) ruptured the cell. With this information, the controller (FIG. 1, 116) can determine a rupture threshold, or strength of a cell. That is, based on sensor (FIG. 1, 110) outputs, the controller (FIG. 1, 116) can determine how far down the flow path the cell gets before rupture occurs. For example, if a fifth sensor (FIG. 1, 110) passes information consistent with an intact cell, but the sixth sensor (FIG. 1, 110) passes information consistent with a ruptured cell, the controller (FIG. 1, 116) determines that six agitation cycles at a certain intensity resulted in cell rupture.

In addition to determining a cell rupture threshold, the controller (FIG. 1, 116) also controls various components of the cell analysis device (420). For example, the component controller (FIG. 4, 430) may independently activate/deactivate certain of the lysing elements (108). As a specific example, a particular lysing element (108) may be activated/deactivated based on lysing results of an earlier lysing element. For example, the component controller (FIG. 4, 430) may activate a downstream lysing element (108) based on the lysing results of an upstream lysing element (108). As a specific example, a second sensor (FIG. 1, 110) may determine that a cell is un-ruptured by the second lysing element (108). Accordingly, the component controller (FIG. 4, 430) may activate the third lysing element (108). The component controller (FIG. 4, 430) may also deactivate a downstream lysing element (108) based on the lysing results of an upstream lysing element (108). As a specific example, a third sensor (FIG. 1, 110) may determine that a cell is ruptured by the third lysing element (108). Accordingly, the component controller (FIG. 4, 430) may deactivate the fourth and subsequent lysing elements (108).

Thus, the example depicted in FIG. 7 with multiple lysing elements (108) in respective lysing chambers (106) facilitates the sequential and gradual increase in intensity of a lysing energy such that a determination of a rupture threshold for different cells can be made at a high resolution.

FIG. 7 also depicts the analysis chamber (114) where lysate analysis occurs. FIG. 7 also depicts the detector (532) that determines when the lysate has been processed. Specifically, in the example depicted in FIG. 7, the detector (532) is external to the analysis chamber (114). As a specific example, in the case of PCR analysis, the detector (532) may be a confocal fluorescent imaging microscope with a single- or multi-band detection capability.

The detector (532) may take many forms. For example, the detector (532) may be an optical scatter sensor, an optical fluorescence sensor, an optical bright field sensing system, an optical dark field sensing system, a thermal property sensor, or an impedance sensor.

FIG. 7 also depicts components that facilitate lysate analysis. For example, the cell analysis device (420) may include at least one chamber (744) fluidically coupled to the analysis chamber (114). The chamber (744) may include a solution, such as a master mix, that is to be mixed with the lysate for cellular evaluation. In this example, the cell analysis device (420) includes a pump (746) per chamber to draw fluid into the at least one analysis chamber (114).

As described above, this pump (746) may be triggered by reception of the lysate in the analysis chamber (114), such that the solution enters the analysis chamber (114) for reaction with the lysate after the lysate has been received in the analysis chamber (114). FIG. 7 also depicts the collector (748) that receives the byproducts. That is, following an analysis operation the resulting solution may be collected. For example, in PCR, a particular nucleic acid may be amplified, the amplified solution may be disposed in the collector (748) and retrieved for subsequent analysis.

Using such a cell analysis device (420), single cell nucleic acid testing can be performed on the fly with enough bandwidth to process a large number of cells in a reasonable amount of time to obtain cell population statistics.

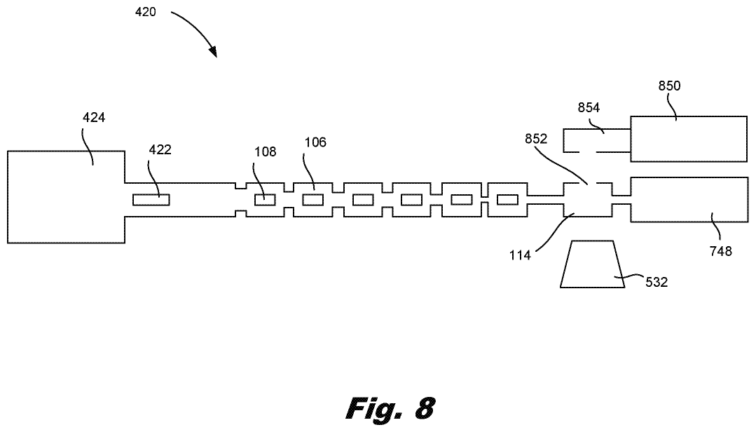

FIG. 8 is a diagram of a cell analysis device (420), according to an example of the principles described herein. Similar to the example depicted in FIG. 7, in this example, the cell analysis device (420) includes a cell reservoir (424), pump (422), multiple lysing chambers (106), lysing elements (108) in each lysing chamber (106), and sensors (FIG. 1, 110) disposed within each lysing chamber (106). This example also depicts the analysis chamber (114), external detector (532), and collector (748).

However, in this example, rather than having the chamber (FIG. 7, 744) be fluidically coupled, and integrated with the cell analysis device (420), the solution is delivered via a different mechanism, for example a different substrate layer. In this example, the analysis chamber (114) includes an orifice (852). Accordingly, the solution, such as a master mix, is injected for example via an ejector. In this example, the cell analysis device (420) includes a port (854) to receive a supply (850). Reagent droplets from a supply (850) received in the port (852), are injected into the analysis chamber (114) via the orifice (852). By using such a port (854), different types of fluids can be introduced into the analysis chamber (114) by switching out supplies (850). For example, a supply (850) with master mix solution is inserted into the port (854) such that PCR may be carried out. Following PCR operations for multiple cells, the master mix supply (850) may be removed and a supply (850) with a washing agent may be inserted to clean out the analysis chamber (114).

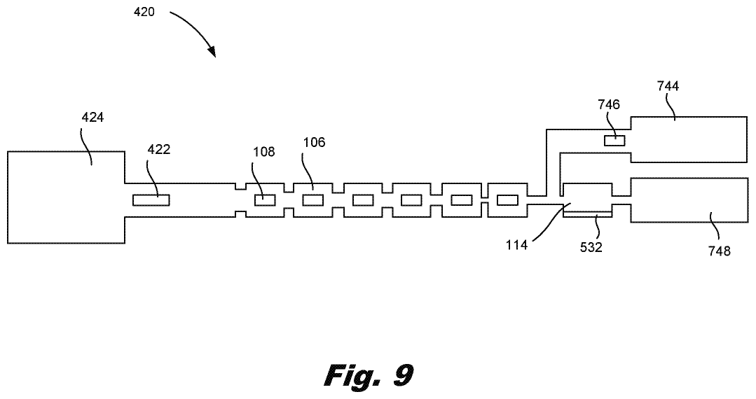

FIG. 9 is a diagram of a cell analysis device (420), according to an example of the principles described herein. Similar to the example depicted in FIG. 7, in this example, the cell analysis device (420) includes a cell reservoir (424), pump (422), multiple lysing chambers (106) with lysing elements (108), and sensors (FIG. 1, 110) per lysing chamber (106). This example also depicts the analysis chamber (114), detector (532), collector (748), chamber (744), and pump (746). However, in this example, rather than having an external detector (532), the detector (532) is embedded in the analysis chamber (114) and is to determine when the lysate has been processed.

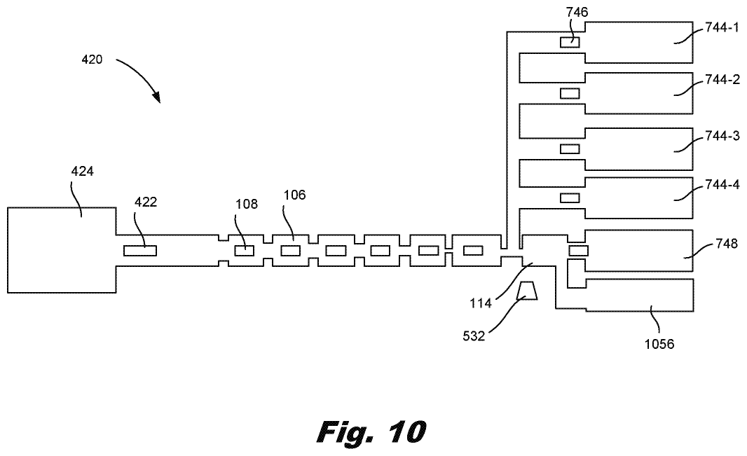

FIG. 10 is a diagram of a cell analysis device (420), according to an example of the principles described herein. Similar to the example depicted in FIG. 7, in this example, the cell analysis device (420) includes a cell reservoir (424), pump (422), multiple lysing chambers (106), with each lysing chamber (106) including a lysing element (108), and sensor (FIG. 1, 110). This example also depicts the analysis chamber (114), detector (532), and collector (748). In the example depicted in FIG. 10, the cell analysis device (420) also includes a waste collector (1056). That is, analyzed lysate may be passed to the collector (748) to be retrieved for further analysis. In this example, the waste fluid, such as for example excess solution and/or carrier fluid, may be collected for disposal in the waste collector (1056).

FIG. 10 also depicts an example where multiple chambers (744-1, 744-2, 744-3, 744-4) and respective pumps (746) are coupled to the analysis chamber (114). For simplicity in FIG. 10, just one instance of a pump (746) is indicated with a reference number.

In this example different chambers (744) may provide different fluids. For example, the first chamber (744-1) and second chamber (744-2) may include different master mix solutions, for example to carry out different nucleic acid tests. The pump (746) of each may be individually activated to draw the corresponding solution into the analysis chamber (114).

Continuing this example, the third chamber (744-3) and fourth chamber (744-4) may include different washing solutions. For example, after a nucleic acid has been amplified, the PCR product may be removed and a wash fluid such as DNAase may be introduced from the third chamber (744-3). Following this washing, a second cleansing solution, such as a DNAease to inactivate the DNAase, may be introduced from the fourth chamber (744-4).

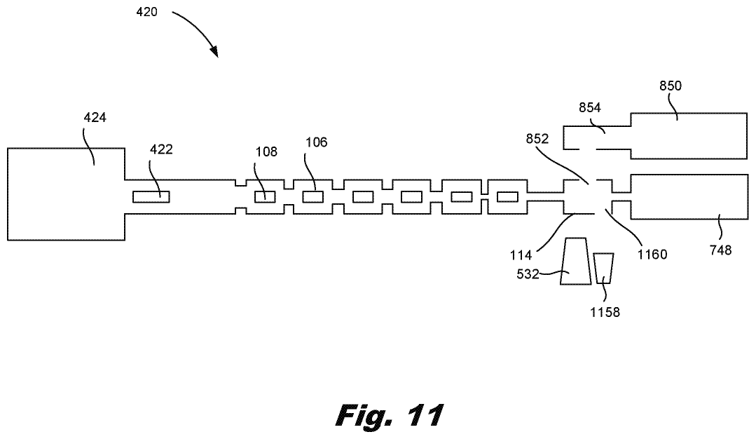

FIG. 11 is a diagram of a cell analysis device (420), according to an example of the principles described herein. Similar to the example depicted in FIG. 7, in this example, the cell analysis device (420) includes a cell reservoir (424), pump (422), multiple lysing chambers (106) with respective lysing elements (108), sensors (FIG. 1, 110), an analysis chamber (114), and a collector (748). In the example depicted in FIG. 11, the cell analysis device (420) includes a port (854) to receive a supply (850) of fluid to be introduced into the analysis chamber (114) via the orifice (852).

However, in the example depicted in FIG. 11, rather than transporting the waste fluid to a waste collector (FIG. 10, 1056), the waste fluid may be ejected through a waste orifice (1160) to a waste receptacle (1158) that may be external to the substrate (FIG. 1, 102) on which the other components are disposed. That is, the cell analysis device (420) may include a waste ejector (1160) to eject waste fluid.

The waste ejector (1160) may include a firing resistor or other thermal device, a piezoelectric element, or other mechanism for ejecting fluid from the firing chamber. For example, the waste ejector (1160) may be a firing resistor. The firing resistor heats up in response to an applied voltage. As the firing resistor heats up, a portion of the fluid in the firing chamber vaporizes to form a bubble. This bubble pushes the waste fluid out the opening and into the waste receptacle (1158). In this example, the waste ejector (1160) may be a thermal inkjet waste ejector (1160).

In another example, the waste ejector (1160) may be a piezoelectric device. As a voltage is applied, the piezoelectric device changes shape which generates a pressure pulse in the firing chamber that pushes a fluid out the waste ejector (116) opening. In this example, the waste ejector (1160) may be a piezoelectric inkjet waste ejector (1160).

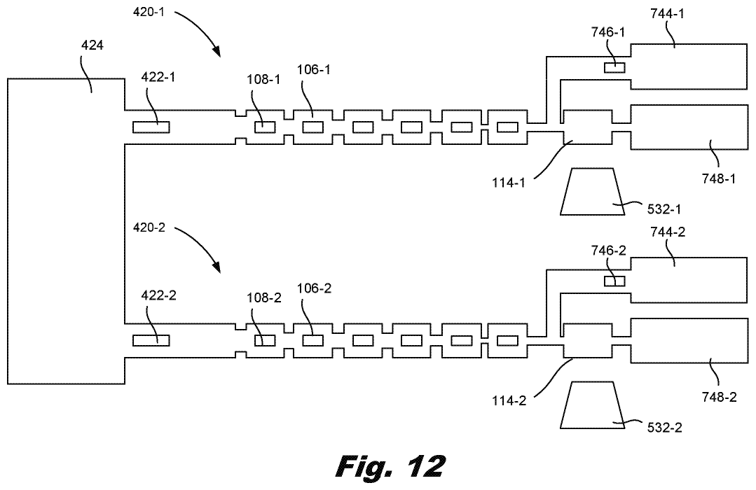

FIG. 12 is a diagram of multiple cell analysis devices (420-1, 420-2), according to an example of the principles described herein. That is, in the example depicted in FIG. 12, the cell analysis system (FIG. 1, 100) may include multiple cell analysis devices (420-1, 420-2), each to analyze an individual cell. In FIG. 12, each cell analysis device (420) and its associated components are distinguished by the “−1” or “−2” designation.

In this example, the multiple cell analysis devices (420) may be in parallel. The multiple parallel cell analysis devices (420) facilitate the processing of more cells. For example, each cell analysis devices (420) analyzes a single cell. Accordingly, multiple parallel cell analysis devices (420) allow multiple cells to be analyzed at the same time, rather than analyzing a single cell at a time. While FIG. 12 depicts multiple cell analysis devices (420-1, 420-2) that implement certain characteristics, i.e., external detectors (532-1, 532-2) and integrated chambers (744-1, 744-2) any of the previously described examples may also be duplicated with parallel cell analysis devices (420-1, 420-2).

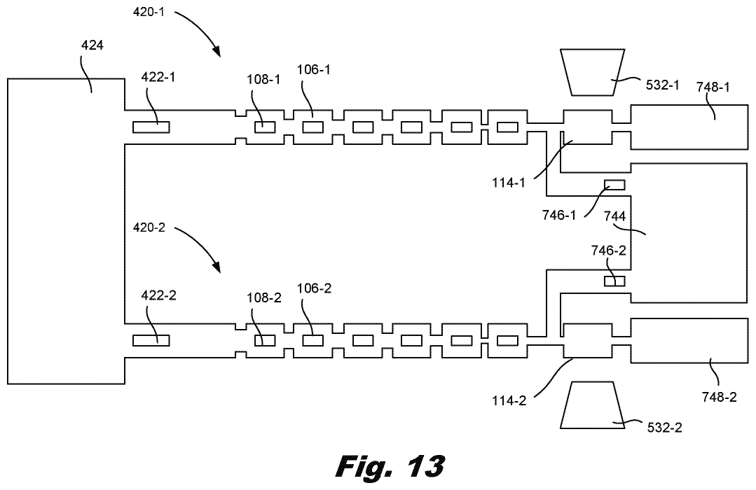

FIG. 13 is a diagram of a cell analysis device (420), according to an example of the principles described herein. As depicted in FIG. 12, the example depicted in FIG. 13 includes multiple cell analysis devices (420-1, 420-2), each to analyze an individual cell. However, rather than each analysis chamber (114-1, 114-2) being supplied with a solution via a unique chamber (744-1, 744-2) as it shown in FIG. 12, in this example, a single solution chamber (744) is fluidically coupled to multiple analysis chambers (114) such that a solution can be provided to the multiple analysis chambers (114) simultaneously. Doing so may simplify the cell analysis device (420) as rather than having multiple smaller chambers (744-2, 744-2) a single larger chamber (744) may be included in the system.

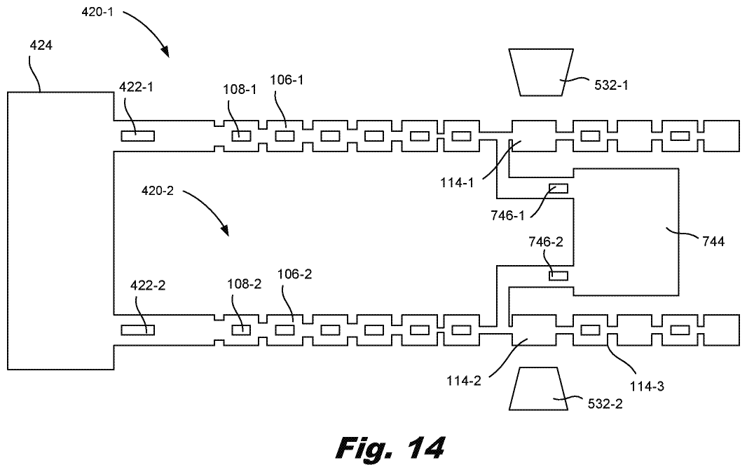

FIG. 14 is a diagram of multiple cell analysis devices (420), according to an example of the principles described herein. As depicted in FIG. 13, the example depicted in FIG. 14 includes multiple cell analysis devices (420-1, 420-2), each to analyze an individual cell and a shared solution chamber (744). However, in the example depicted in FIG. 14, there are multiple additional analysis chambers (114-3). For simplicity in FIG. 14, just one additional analysis chamber (114-3) along one path is indicated with a reference number.

Each additional analysis chamber (114-3) may be used for a different type of analysis. That is, any number of analysis chambers (114-3) may be used to carry out any number of subsequent analyses. In some example, the analysis chambers (114) include ejectors to carry out the subsequent analysis. For example, some of the analysis chambers (114) may include ejectors that align with predetermined spots on a surface. As a specific example, the configuration of ejectors in the additional analysis chambers (114-3) may align with wells on a micro well plate. In this example, different wells on the plate include different antibodies that chemically react with certain proteins. Different sub-samples of the lysate are ejected into the different wells. If the lysate includes a particular protein that binds with an antibody included in that well, a chemical reaction is triggered, which may be visual to an operator. That is, in this example, the additional analysis chambers (114-3) may be used for lysate protein identification. While specific reference is made to one particular type of additional analysis, a variety of other analyses may be executed in the additional analysis chambers (114-3).

In summary, using such a cell analysis system 1) provides for effective monitoring of cell lysis on a per-cell basis; 2) ensures sufficient lysis without degradation to cell contents; 3) provides control of the amount of analyte to be delivered downstream; 4) identifies subsets of cell population that are difficult to lyse; 5) provides a feedback signal for automated control of the lysis operation, 6) allows combined cell analysis, i.e., a genetic analysis and a mechanical property analysis; 7) allows for an entire preparation and analysis operation on a single substrate; 8) can be integrated onto a lab-on-a-chip; 9) is scalable and can be parallelized for high throughput, 10) is low cost and effective, and 11) reduces the contamination of the sample.

What is claimed is:

1. A cell analysis system, comprising:
a substrate;
a feedback-controlled lysis system formed in the substrate to rupture a cell membrane, the feedback-controlled lysis system comprising:
at least one lysing chamber to receive a single cell to be lysed;
a lysing element to agitate the single cell; and
a sensor to detect a state within the lysing chamber;
a microfluidic channel formed in the substrate to:
serially feed individual cells from a volume of cells to the feedback-controlled lysis system; and
deliver a lysate of a ruptured cell to at least one analysis chamber;
at least one analysis chamber formed in the substrate to process the lysate; and
a controller to determine when a cell membrane has ruptured.

2. The cell analysis system of claim 1, further comprising a detector that is external to the analysis chamber to determine when the lysate has been processed.

3. The cell analysis system of claim 1, further comprising a detector that is embedded in the analysis chamber to determine when the lysate has been processed.

4. The cell analysis system of claim 1, further comprising:
an orifice in the analysis chamber; and
a port to receive a supply which holds a fluid to be injected into the analysis chamber.

5. The cell analysis system of claim 1, further comprising:
at least one chamber fluidically coupled to the analysis chamber; and
a pump per chamber to draw fluid into the at least one analysis chamber.

6. The cell analysis system of claim 5, wherein the at least one chamber comprises a single chamber fluidically coupled to multiple analysis chambers.

7. The cell analysis system of claim 1, further comprising a collector formed in the substrate to collect analyzed lysate.

8. The cell analysis system of claim 1, further comprising a waste ejector to eject waste fluid.

9. A method, comprising:
receiving, at a feedback-controlled lysis system of a cell analysis system, a cell to be lysed;
activating the feedback-controlled lysis system to agitate the cell to be lysed;
detecting when a cell membrane is ruptured;
responsive to a detection that the cell membrane is ruptured, transporting a lysate of the cell to an analysis chamber; and
performing an analysis operation on the lysate.

10. The method of claim 9, further comprising determining when the analysis operation is complete.

11. The method of claim 9, further comprising controlling components of the cell analysis system based on contents of the analysis chamber.

12. The method of claim 9, wherein detecting when a cell membrane is ruptured comprises:
measuring a conductivity within the lysing chamber;
analyzing the conductivity within the lysing chamber to determine that the cell membrane has ruptured; and
when the cell is un-lysed, re-lysing the cell.

13. The method of claim 9, further comprising incrementally adjusting an intensity of lysis until the cell membrane ruptures.

14. A cell analysis system, comprising:
a substrate;
at least one cell analysis device formed in the substrate, each cell analysis device comprising:
a feedback-controlled lysis system to rupture a cell membrane, the feedback-controlled lysis system comprising:
at least one lysing chamber to receive a single cell to be lysed;
a lysing element to agitate the single cell; and
a sensor disposed within the lysing chamber to detect a state within the lysing chamber;
a microfluidic channel to:
serially feed individual cells from a volume of cells to a feedback-controlled lysis system; and
deliver a lysate of a ruptured cell to at least one analysis chamber;
at least one analysis chamber to process the lysate; and
a cell reservoir to hold a volume of cells;
a pump to move fluid through the cell analysis system; and
a controller disposed on the substrate to analyze the cell, the controller comprising:
a lysate analyzer to analyze properties of a lysate of the cell;
a rupture analyzer determines the rupture threshold of the cell based on parameters of a cycle when a cell membrane ruptures; and
a component controller to alter operation of at least one component of the analytic device based on a cell rupture.

15. The cell analysis system of claim 14, wherein the at least one analysis chamber comprises multiple analysis chambers.
